(19) United States
(12) Patent Application Publication
(10) Pub. No.: US 2005/0129694 A1
(43) Pub. Date: Jun. 16, 2005

(54) Pharmaceutical composition for the prophylaxis and/or treatment of virus diseases

(76) Inventors: Stephan Ludwig, Wurzburg (DE); Oliver Planz, Rottenburg (DE); Hans-Harald Sedlacek, Marburg (DE); Stephan Pleschka, Giessen (DE)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/486,313

(22) PCT Filed: Jul. 26, 2002
(86) PCT No.: PCT/DE02/02810

(30) Foreign Application Priority Data
Aug. 8, 2001 (DE) 101389124

(51) Int. Cl. A61K 39/395; A61K 31/553; A61K 31/551

(52) U.S. Cl. 424/146.1; 514/211.08; 514/212.04; 514/275; 514/44; 514/616; 514/341; 514/469; 514/221

(57) ABSTRACT

The invention relates to the use of at least one active substance for producing a pharmaceutical composition for the prophylaxis and/or treatment of at least one viral disease. It is characterized by active substance(s) which inhibit(s) either at least two kinases or at least one SEK kinase of a cellular signal transmission path such that virus multiplication is inhibited.

### FIELD OF THE INVENTION

The invention relates to the use of at least one active substance for producing a pharmaceutical composition for the prophylaxis and/or treatment of at least one virus disease, and to a combination preparation herefor.

### BACKGROUND OF THE INVENTION AND PRIOR ART

Infections with RNA or DNA viruses are a significant threat for the health of man and animal. For instance, infections with influenza viruses do still belong to the big epidemics of mankind and cause year for year a big number of casualties. In terms of the national economies, they are an immense cost factor, for instance due to unfitness for work. Infections with the Borna disease virus (BDV), which mainly affects horses and sheep, but which has also been isolated for humans and is connected to neurological diseases, equally have an enormous economic importance.

The problem of controlling in particular RNA viruses is the adaptability of the viruses caused by a high fault rate of the viral polymerases, which makes the production of suitable vaccines as well as the development of antiviral substances very difficult. Furthermore it has been found that the application of antiviral substances immediately directed against the functions of the virus, shows a good antiviral effect at the beginning of the treatment, but will quickly lead to the selection of resistant variants based on mutation. An example is the anti-influenza agent amantadine and its derivatives directed against a transmembrane protein of the virus. Within a short time after the application, resistant variants of the virus are generated. Other examples are the new therapeuticals for influenza infections inhibiting the influenza-viral surface protein neuraminidase. To these belongs for instance Relenza. In patients, Relenza-resistant variants have already been found (Gubareva et al., J Infect Dis 178, 1257-1262, 1998). Hopes placed in this therapeutical could therefore not be fulfilled.

Because of the in most cases very small genomes and thus limited coding capacity for functions being necessary for the replication, all viruses are dependent to a high degree from functions of their host cells. By exertion of influence on such cellular functions being necessary for the viral replication, it is possible to negatively affect the virus replication in the infected cell. Herein, there is no possibility for the virus to replace the lacking cellular function by adaptation, in particular by mutations, in order to thus escape from the selection pressure. This could already be shown for the influenza A virus with relatively unspecific inhibitors against cellular kinases and methyl transferases (Scholtissek and Müller, Arch Virol 119, 111-118, 1991).

It is known in the art that cells have a multitude of signal transmission paths, by means of which signals acting on the cells are transmitted into the cell nucleus. Thereby the cell is capable to react to external stimuli and to react by cell proliferation, cell activation, differentiation, or controlled cell death. It is common to these signal transmission paths that they contain at least one kinase activating by phosphorylation at least one protein subsequently transmitting a signal. When observing the cellular processes induced after virus infections, it is found that a multitude of DNA and RNA viruses preferably activate in the infected host cell a defined signal transmission path, the so-called Raf/MEK/ERK kinase signal transmission path (Benn et al., J Virol 70, 4978-4985, 1996; Bruder and Kovesdi, J Virol 71, 398-404, 1997; Popik and Pitha, Virology 252, 210-217, 1998; Rodems and Spector, J Virol 72, 9173-9180, 1998). This signal transmission path is one of the most important signal transmission paths in a cell and plays a significant role in proliferation and differentiation processes. Growth factor-induced signals are transmitted by successive phosphorylation from the serine/threonine kinase Raf to the dual-specific kinase MEK (MAP kinase kinase/ERK kinase) and finally to the kinase ERK (extracellular signal regulated kinase). Whereas as a kinase substrate for Raf, only MEK is known, and the ERK isoforms were identified as the only substrates for MEK, ERK is able to phosphorylate a whole number of substrates. To these belong for instance transcription factors, whereby the cellular gene expression is directly influenced (Cohen, Trends in Cell Biol 7, 353-361, 1997; Robinson and Cobb, Curr. Opin. Cell Biol 9, 180-186, 1997; Treisman, Curr. Opin. Cell Biol 8, 205-215, 1996). The investigation of the role of this signal transmission path in cellular decision processes has led to the identification of several pharmacological inhibitors, which inhibit the signal transmission path, among other positions, on the level of MEK, i.e. relatively close to the beginning of the signal transmission path (Alessi et al., J Biol Chem 270, 27489-27494, 1995; Cohen Trends in Cell Biol 7, 353-361, 1997; Dudley et al., PNAS USA 92, 7686-7689, 1995; Favata et al., J Biol Chem 273, 18623-18632, 1998).

Newer data show that the inhibition of the Ras/Raf/MEK/ERK signal transmission path can drastically inhibit the intracellular multiplication of intranuclearly replicating negative strand viruses, for instance influenza A virus and Borna disease virus (BDV), by active substances, which inhibit relatively selectively one of the kinases involved in this signal transmission path, in particular MEK (Pleschka et al., Nature cell Biol 3, 301-305, 2001; Planz et al., J Virol 10, 4871-4877, 2001).

The drawback of prior art antiviral active substances is that they are either directed against a viral component and thus quickly lead to resistances (cf. amantadine), or act in a too broad and unspecific manner against cellular factors (for example methyl transferase inhibitors), and significant side effects are to be expected. Consequently, none of the substances being active against cellular factors is known up to now to have been developed to a therapeutical for virus diseases. On the other hand, the inhibition of other kinases, for instance the inhibition of the kinase JNK of the MEKK/SEK/JNK signal transmission path, can increase the virus multiplication.

Further it is known that the increased activation of again other kinases, for instance of the protein kinase C (PKC), inhibits the replication of viruses (Driedger and Quick, WO 92/02484).

Up to now there was the assumption that negative strand RNA viruses only use the Raf/MEK/ERK signal transmission path for their multiplication, and that thus an inhibition of the virus multiplication for this signal transmission path, in particular by an inhibition of MEK, leads to a complete inhibition of the virus multiplication. Since in a cell the signal transmission paths do however not comprise a function being closed in itself, but further signal transmission paths are in addition activated to different extents by an activation of the one signal transmission path via cross-linkings, it cannot be excluded, in principle, that the Raf/MEK/ERK signal transmission path can be by-passed by the cell as well as by the viruses, and the therapeutic effect of an active substance inhibiting a virus multiplication for the Raf/MEK/ERK signal transmission path could be limited hereby.

### TECHNICAL OBJECT OF THE INVENTION

Therefore, there is an enormous need for finding and using antiviral active substances, which act in an additional or complementing manner to those inhibiting the virus multiplication for the Raf/MEK/ERK signal transmission path. Finding such active substances is made difficult by that for instance inhibitors of kinases outside the Raf/MEK/ERK signal transmission path, for instance inhibitors of the kinase JNK, can promote a virus multiplication, i.e. have the opposite effect to the desired one (Ludwig et al., J Biol Chem 276, 1-9, 2001), and that the activation of selected kinases, for instance PKC, by substances may act in an antiviral manner.

### BASICS OF THE INVENTION AND PREFERRED EMBODIMENTS

A subject matter of the present invention is the use of at least active substance for the prophylaxis and/or treatment of at least one virus disease, the active substance(s) acting on at least two kinases of a cellular signal transmission path such that a virus multiplication is substantially inhibited or substantially inhibits an SEK kinase. Subject matter of the present invention is further the combination of at least one active substance according to the invention with at least one further antivirally active substance, preferably not being a kinase inhibitor, for the prophylaxis and/or treatment of a virus disease.

Surprisingly it has now been found that the inhibition of an SEK kinase in the MEKK/SEK/JNK signal transmission path inhibits the virus multiplication. This is surprising since the inhibition of JNK in the MEKK/SEK/JNK signal transmission path causes the opposite effect, an increased virus multiplication (Ludwig et al., J Biol Chem 276, 1-9, 2001). Since the JNK/AP-1 signal path is significantly involved in the virus-induced expression of IFNβ, a strongly antivirally effective cytokine, the lacking IFNβ expression after inhibition of JNK seems to permit an increased virus multiplication. Surprisingly it has been found, further, that an inhibition of at least two kinases of a cellular signal transmission path by at least one active substance inhibits the virus multiplication to a much higher degree than in the case that only one kinase is inhibited by an active substance. For instance, the inhibition of p38 and MAPKAPK3 or the inhibition of MKK6 and p38 or the inhibition of Raf and MEK leads to a distinctly stronger antiviral effect than the inhibition of only one kinase, in particular of the kinase activating (the second kinase). Surprisingly, it has been found, further, that the inhibition of at least one kinase of a cellular signal transmission path by at least one active substance and the additional administration of an antiviral active substance, which does not represent an inhibitor of a kinase, leads to a strong inhibition of the virus multiplication. This inhibition by the combination was stronger than could be expected for the addition of the single effects of the components of this combination. For instance, cells were infected with influenza A, and the cells were treated with the kinase inhibitor U0126, which is known to inhibit in the Raf/MEK/ERK signal transmission path the MEK kinase, and simultaneously with amantadine. It is known that amantadine is not effective for all influenza A viruses and not for influenza B viruses, furthermore that it leads in a short time to the generation of resistant virus variants. The combination of U0126 and amantadine led to an inhibition of the multiplication of influenza A viruses, and the combination may act in a stronger way than the single components alone.

To these cellular signal transmission paths belong for instance: MEKK2,-3/MEK5/ERK5; Raf/MEK/ERK; MEKK/SEK/JN; JAK1, JAK2, JAK3, TYK2/hetero and homodimers of JAK1,-2,-3, TYK2; ASK/MKK3,-6/p38; ASK/MKK4,-7/JNK; MEKK4/MKK4,-7; DLK/MKK4,-7; Tpl-2/MKK4,-7; Tpl-2/MEK5/ERK5; MLK-3/MKK3, -6; MLK-3/MKK4,-7; TAK/NIK/IKK; TAK/MKK3,-6; TAK/MKK4,-7; PAK/MKK3,-6; PAK/IKK; Cot,Tpl-2/IKK; PKC/IKK; PKB/IKK; PKC/Raf; PAK/Raf; Lck/Raf; MEKKs/IKK; PI3K/PDK1/PKB; JAK/TYK/PLCgamma; JAK/Tyk/stat complexes; MAP kinases/MAPKAP kinases, for instance: ERK/3pK, ERK/Rskp90, p38/MAPKAP kinases, p38/3pK.

Details for these signal transmission paths have been described by Sedlacek (Drug 59, 435-476, 2000) and in various other publications (Karin and Ben-Neriah, Ann Rev Immunol 18, 621-663, 2000; Vertegaal et al., Cell Signal 12, 759-768, 2000; Vanhaesebroeck and Alessi, Biochem J 346, 561-576, 2000; Ludwig et al., Mol Cell Biol 16, 6687-6697, 1996; New et al., J Biol Chem 274, 1026-1032, 1999; Ni et al., Biochem Biophys Res Commun, 243, 492-496, 1998; Rebecchi and Pentyala, Physiol Rev 80, 1291-335, 2000; Cohen, Trends in Cell Biol 7, 353-361, 1997; Robinson and Cobb, Curr Opin Cell Biol 9, 180-186, 1997; Rane and Reddy, Oncogene, 19, 5662-5679, 2000; Sun et al., Curr Biol. 10, 281-284, 2000; Garrington and Johnson, Curr Opin Cell Biol 11, 211-218, 1999).

An active substance in the meaning of the present invention is a substance, which is capable to either act on at least one kinase of a cellular signal transmission path such that a virus multiplication is substantially inhibited, or to substantially inhibit an SEK kinase of a cellular signal transmission path. Further, active substances in the meaning of the present invention are derivatives of the active substances, which are for instance transformed by enzymatic cleavage into an active substance according to the invention. Active substances in the meaning of the present invention are in addition pre-stages of active substances, which are metabolically transformed into an active substance according to the invention. To the active substances in the meaning of the present invention belong for instance: kinase-inhibiting flavone derivatives or benzopyran derivatives; kinase inhibiting derivatives of the 4H-1-benzopyran, for instance as disclosed in EP 0137193 and EP 366061. The structural interrelation for these derivatives has been described in detail for instance by Sedlacek et al. (Int J Oncol 9, 1143-1168, 1996); flavopiridol derivatives, for instance disclosed by Kim et al., J Med Chem 43(22), 4126-4134, 2000, in particular thio-flavopiridol and oxy-flavopiridol; 2-(2-amino-3-methoxyphenyl)-4-oxo-4H-(1) benzopyran, for instance disclosed by Ebendal (WO 00/50030); 7,12-dihydroindolo(3,2-d)(1)benzazepin-6(5H)-one (NSC 664704 Sausville et al. Pharmacol. Ther. 82(2-3), 285-292, 1999); phosphokinase inhibitors, for instance 70H-staurosporine and their phosphokinase-inhibiting derivatives, butyrolactones, roscovitines, purvalanol A, emodin, anilinoquin-azolines (PD-168393 and PD 169414), PD 184352 (Duesbery et al., Nature Medicine 5(7), 736-737, 1999) and phenylamino-pyrimidines (STI 571, CGP 78850, CP 358774, CP59326 and CGP 60474)), trioylimidazole (1-779450) (Meijer et al., Parmacol. Ther. 82(2-3), 297-284, 1999; Sedlacek et al., Int J Oncol 9, 1143-1168, 1996; Sedlacek Drug 59(3), 435-476, 2000); paullones (Zaharevitz et al., Cancer Res. 59, 2566-2569, 1999); SB203580 [4-(4-fluorophenyl)-2-(4-methylsulfinylphenyl)-5-(4-pyridyl)1H-imidazole, (Ishikawa et al., J Neurochem 75(2), 494-502, 2000, Harada and Sugimoto, Jpn J Pharmacol 79(3), 369-378, 1999)]; kinase-inhibiting butadiene derivatives, in particular derivatives of diaminodicyano-(R)-thiobutadiene; UO126 [1,4-diamino-2,3-dicyano-1,4-bis(2aminophenylthio) butadiene] (Favata et al., J Biol Chem 273(29), 18623-18632, 1998; DeSilva et al., J Immunol 160, 4175-4181, 1998)]; PD098059 [2-2′-amino-3′-methoxyphenyl)-oxanaphthalene-4-on)], (Dudley et al., PNAS USA 92, 7686-7686, 1995); PD184352 [2-(2-chloro-4-iodo-phenylamino)-N-cyclo-propylmethoxy-3,4-difluorobenzamide], (Sebolt-Leopold et al., Nature Med 5, 810-816, 1999); 3-aminomethylene-indoline derivatives (Heckel et al., DE 92 4401, Eberlein et al., DE 84 4003; WO 00/018,734); CEP-1347 (KT7515) bis-ethylthiomethyl (Maroney et al., J Neurochem 73(5), 1901-1912, 1999); tetrapyrrolic macrocycles, for instance 5,10,15,20-tetraarylporphyrin and 5,10,15-triarylcorrol (Aviezer et al., WO 00/27379); pyrimidone derivatives (Ando et al., WO 00/018,758); 3-aminomethylene-indoline derivatives (Heckel et al., DE 92 4401, Eberlein et al., DE 84 4003; WO 00/018,734); pyrazolo (3,4-b) pyridine derivatives (Bursuker et al., WO 99/30710); pyrazole derivatives (Anantanarayan et al., WO 00/031,063); 1,4-substituted piperidine derivatives (Caravatti et al., EP 374095); lipoid ammonium salts (Daniel et al., WO 90/04918); 4-(4-fluorophenyl)-2-(4-hydroxyphenyl)-5-(4-pyridyl)1H-imidazole (SB202190); SL 327 (a butadiene derivative); dominant negative mutants of a kinase of a cellular signal transmission path; antisense oligonucleotides, which specifically accumulate at the DNA sequence or mRNA sequence, coding for a kinase of a cellular signal transmission path and inhibit the transcription or translation thereof; dsoligonucleotides, which are suitable for a specific degradation of the mRNAs of kinases of a cellular signal transmission path by the RNAi technology according to the method as described by Tuschl et al (Genes Dev 13: 3191-3197, 1999) and by Zamore et al (Cell 101: 25-33, 2000); antibodies or antibody fragments specific for a kinase of a cellular signal transmission path, or fusion proteins, containing at least one antibody fragment, for instance an Fv fragment, which inhibit the kinase activity of at least one kinase; peptides, which inhibit the interaction of at least two kinases of a cellular signal transmission path.

Preferably, at least one active substance according to the invention is used for a virus disease caused by RNA or DNA viruses, preferably negative strand RNA viruses, for instance influenza viruses, or Borna viruses.

Another embodiment of the present invention represents a combination preparation for the prophylaxis and/or treatment of at least one virus disease, containing at least two active substances which act either on at least two kinases of a cellular signal transmission path such that a virus multiplication is substantially inhibited, or substantially inhibit an SEK kinase, preferably selected from the above active substances, the combination preparation being applicable in the form of a mixture or as individual components at the same time or at different times at the same positions or at different positions.

By the administration of a combination preparation, containing at least two different active substances, the virus multiplication is inhibited by acting on at least two kinases of a cellular signal transmission path. The administration of the combination preparation can be made as a mixture of the active substances. The active substances can however also be separately administered at the same position, for instance intravenous, or also at separate positions, at the same time or at different times within a period of time, in which the substance administered first is still effective, for instance in a period of time of three days.

Another embodiment of this invention represents the administration of a combination preparation of at least one active substance according to the invention and at least one antiviral active substance not representing a kinase inhibitor. To these antiviral active substances belong for instance: 1-adamantanamine (amantadine); rimantadine; neuraminidase inhibitors such as for instance Relenza; synthetic nucleoside analogs such as for instance 3-deazaadenosine and ribavirin.

Another embodiment of the present invention relates to a test system for finding active substances, which inhibit either on at least two kinases of a cellular signal transmission path or on an SEK kinase such that a virus multiplication is substantially inhibited, containing: a. at least one cell infectable with at least one virus, said cell containing either at least two kinases of a cellular signal transmission path or at least one SEK kinase and at least one virus infecting the cells, or b. at least one cell infected with at least one virus, said cell containing either at least two kinases of a cellular signal transmission path or at least one SEK kinase.

Cells in the meaning of the present invention are cells from different organs and tissues, for instance cells of the blood and lymphatic vessels, cells, which coat body cavities. Further are comprised cell cultures, in particular those, which can be purchased from cell collections, for instance the ATCC, in particular permissive eukaryotic cell cultures, for instance: 293, 293T and 293T7 (homo sapiens); B82, NIH 3T3, L929 from mus musculus; BHK from cricetus cricetus; CHO from cricetulus griseus; MDCK from canis familiaris; vero, COS-1 and COS-7 from cercopithecus aethiops; and primary embryo fibroblasts from gallus gallus (CEF cells).

It is for instance tested, in the test system according to the invention for finding active substances, by addition of substances, preferably in concentrations of 0.001 μmol to 100 μmol, and viruses in a particle number, which can infect the selected cell, whether a substance is capable to inhibit the virus multiplication, without damaging the cell.

Preferably, the virus used in the test system according to the invention is an RNA or DNA virus, preferably an influenza virus.

In a preferred embodiment, the cell of the test system according to the invention contains at least one overexpressed kinase, in particular by introduction of one or several genes coding the kinase(s). By this overexpression, substances are detected, which strongly inhibit kinases as well as can intercellularly reach high concentrations for the inhibition of the overexpressed kinase. As a control mechanism, the expression for at least one kinase is inhibited in a cell of the test system according to the invention, for instance: by the introduction of an antisense DNA or an antisense RNA; by the introduction of at least one gene coding for at least one dominant-negative mutant of at least one superordinated kinase; and/or by the introduction of at least one gene coding for at least one dominant-negative mutant of at least one subordinated kinase of a signal transmission path.

Another embodiment of the present invention relates to a method for finding at least one active substance for the prophylaxis and/or treatment of virus diseases, which substantially inhibits or inhibit the virus multiplication during virus diseases, comprising the following steps: a. bringing at least one test system according to the invention into contact with at least one potential active substance, and b. determining the effect on the virus multiplication.

Bringing into contact in the meaning of this invention may for instance take place by addition of the active substances to the nutrient medium of a cell culture or by local or systemic administration of the active substances to an organism. Bringing into contact in the meaning of the present invention also comprises the methods usual in the prior art, which permit the introduction of substances into intact cells, for instance infection, transduction, transfection and/or transformation and other methods known to the man skilled in the art. These methods are in particular preferred, if the substance is represented by viruses, naked nucleic acids, for instance antisense DNA and/or antisense RNA, viroids, virosomes and/or liposomes, virosomes and liposomes being equally suitable to introduce, in addition to a nucleic acid molecule, further active substances into the cell.

The determination of the effect on the virus multiplication is for instance made by plaque assays by comparison of virus titers of infected and not infected cells.

Another preferred embodiment of the present invention relates to a method for producing a drug for the prophylaxis and/or treatment of at least one virus disease, which substantially inhibits the virus multiplication during virus diseases, comprising the following steps: a. performing a test system according to the invention, and b. reacting the found active substance(s) with at least one auxiliary and/or additional substance.

Preferably, the active substance according to the present invention for the local or systemic administration to an organism is made up to a drug by means of the methods and auxiliary and/or additional substances known to the man skilled in the art.

Suitable auxiliary and additional substances, which serve for instance for the stabilization or conservation of the drug or diagnosticum, are generally known to the man skilled in the art (see for instance Sucker H et al. (1991) Pharmazeutische Technologie, 2^{nd} edition, Georg Thieme Verlag, Stuttgart). Examples for such auxiliary and/or additional substances are physiologic common salt solutions, Ringer's dextrose, dextrose, Ringer's lactate, demineralized water, stabilizers, antioxidants, complex-forming agents, antimicrobial compounds, proteinase inhibitors and/or inert gases.

The local administration may for instance be made on the skin (for instance with a transdermal system), on the mucous membrane, into a body cavity, into an organ (for instance i.m.), into a joint or into the connective tissue or the stroma. The systemic administration is preferably made into the blood circulation, for instance i.v., into the peritoneal cavity or into the abdominal cavity.

The making-up of the drug containing the active substance according to the invention depends on the type of the active substance and on the type of the administration thereof and may for instance be a solution, a suspension, an ointment, a powder, a spray or another inhalation preparation. Preferably, nucleotide sequences are inserted by means of methods known to the man skilled in the art into a viral vector or a plasmid and reacted with auxiliary substances for the cell transfection. To these auxiliary substances belong for instance cationic polymers or cationic lipids. Antisense oligonucleotides are derivatized with the methods well known to the man skilled in the art in order to protect them from the enzymatic degradation by DNAs or RNAs.

The active substance according to the invention can be provided in the form of a salt, ester, amide, or as a pre-stage, and preferably only those modifications of the active substance are employed, which do not cause an excessive toxicity, irritations or allergic reactions of the patient.

The active substance is mixed under sterile conditions with a physiologically acceptable carrier substance and potential preservatives, buffers or drivers, as may be required. Such carrier substances for drug preparations are known to the man skilled in the art.

Preferably the active substance according to the invention is administered in a single dose, particularly preferably in several doses, and the individual doses do not exceed the maximum tolerated dose (MTD) of the respective active substance for man. Preferably, a dose is selected, which is half the MTD. For instance for the infusion of flavopiridol, the MTD for the tumor patient is 50 mg/m²/dx3 (Senderowicz et al J Clin Oncol 16(9):2986-2999, 1998). For the application to man in the meaning of the present invention, flavopiridol is thus to be administered in a daily dose of 0.1-50 mg/m², preferably 5-30 mg/m², particular preferable 25 mg/m². The daily dose can be administered as a one-off dose per day or in several partial portions distributed over the day, preferably in approximately identical time intervals.

According to the present invention, the administration may be made either locally or systemically, only on one day or over a couple of days daily or on every second or third day over several weeks, until a therapeutic effect can be observed.

Further variants of the invention are described in claims 17 to 19, and the above and below statements will apply here in an analogous manner.

### EXAMPLE 1

### Positive Control I (Influenza A Virus)

For the multiplication of the influenza A viruses, permissive, eukaryotic cell cultures (madine darby canine kidney (MDCK) cells) are washed with a physiologic salt solution in parallel batches having identical cell counts according to methods generally being usual for cell cultures, and are infected with the same amount of the infectious influenza A virus strain WSN-HK (reassortant with seven gene segments of the influenza strain A/WSN/33 and the NA gene of the influenza strain A/HK/8/68) in a ratio of 0.0025 infectious virus particle per cell for one hour at room temperature. 30 min before the infection, the MDCK cells are incubated in a suitable cell culture medium, which is reacted for a positive control in different concentrations with the kinase inhibitor U0126 [1,4-diamino-2,3-dicyano-1,4-bis(2aminophenylthio)butadiene] (0 μM, 30 μM, 40 μM, 50 μM dissolved in DMSO) at 37° C. and 5% CO₂ concentration. As a solvent control, MDCK cells are incubated with cell culture medium, which is reacted with corresponding different amounts of DMSO. During the infection, the kinase inhibitor U0126 is added to the inoculum, or as a solvent DMSO, in the corresponding concentrations. Subsequently, the inoculum is removed, and the infected cells are incubated in a suitable cell culture medium, which is reacted in different concentrations with the kinase inhibitor U0126 (0 μM, 30 μM, 40 μM, 50 μM dissolved in DMSO), for 48 h, at 37° C. and 5% CO₂ concentration. As a solvent control, infected MDCK cells are incubated with cell culture medium, which is reacted with corresponding different amounts of DMSO. 24 hours after the infection, 200 μl of the medium supernatant are removed, and the same volume of inhibitor or DMSO-containing cell culture medium is added. After 48 hours, again a sample is taken. The cell culture supernatants of the respective samples for the 24 and the 48-hours value are tested according to usual virological methods for the amount of haemagglutinating units (HA titer) representing the total production of virus particles, and for the amount of newly formed infectious virus particles (plaque assay on MDCK cells). As a result, it can be stated in such an experimental approach that with increasing concentration of the kinase inhibitor U0126 in the cell culture medium, a significant reduction (approx. 80% for 50 μM U0126) of the number of newly formed infectious virus particles, compared to the control reaction without inhibitor U0126 or to the solvent controls will take place. The macroscopic investigation of MDCK cells, which were treated with corresponding concentrations of DMSO or inhibitor U0126 dissolved in DMSO, as well as a cytotoxicity investigation by means of propidium iodide staining, show that neither solvent nor inhibitor have a significant cytotoxic effect on the cells.

### EXAMPLE 2

### Positive Control II (Influenza B Virus)

This example shows that for a concentration of 60 μM of the MEK inhibitor U0126 in the cell culture medium, also the number of newly formed infectious influenza B virus particles is significantly reduced. For the multiplication of influenza B viruses, permissive, eukaryotic cell cultures (madine darby canine kidney (MDCK) cells) are washed with a physiologic salt solution in parallel batches having identical cell counts according to methods being generally usual for the cell culture, and are infected with the same amount of the infectious influenza B virus strain Massachusetts/6/93 in a ratio of 0.01 infectious virus particle per cell for one hour at room temperature. After the infection, the inoculum is removed, and the infected cells are incubated in a suitable cell culture medium (contains 2 μg/ml trypsin), which is reacted with the MEK inhibitor U0126 (60 μM dissolved in DMSO), for 60 h at 37° C. and 5% CO₂ concentration. As a control, infected MDCK cells are incubated with cell culture medium, which is reacted with the corresponding amount of DMSO. Every 12 hours after the infection, samples of the medium supernatant are taken. The respective samples of the cell culture supernatants are tested according to usual virological methods for the amount of newly formed infectious virus particles (plaque assay on MDCK cells). As a result, it can be stated in such an experimental approach that with the corresponding concentration of the MEK inhibitor U0126 in the cell culture medium, a significant reduction (approx. 90%) of the number of newly formed infectious virus particles, compared to the control reaction (solvent control without MEK inhibitor U0126) will take place. The macroscopic investigation of MDCK cells, which were treated with corresponding concentrations of DMSO or MEK inhibitor U0126 dissolved in DMSO, as well as a cytotoxicity investigation by means of propidium iodide staining, show that neither solvent nor inhibitor have a significant cytotoxic effect on the cells.

### EXAMPLE 3

### Specific Inhibition of the Virus Multiplication for the Cellular MEKK/SEK/JNK Signal Transmission Path

The influence of a specific inhibition of SEK/MKK4 was firstly investigated by means of the transient transfection of MDCK cells with a dominant-negative mutant of the kinase SEK KD. In the case of this mutant, a lysine amino acid residue at amino acid position 129 was transformed by specific mutagenesis on the DNA level into an arginine amino acid residue (Ludwig et al. Mol Cell Biol 16, 6687-6697, 1996), a mutation, which disturbs the ATP binding of the kinase, and the kinase thus exists in an inactive form. This mutant is in case of an overexpression dominant-negative over the endogenous wild type (Ludwig et al. Mol Cell Biol 16, 6687-6697, 1996). For the experiment, MDCK cells were transfected with the pEBG empty vector or the pEBG SEK KD expression construct (Ludwig et al. Mol Cell Biol 16, 6687-6697, 1996) with the assistance of the transfection reagent Lipofectamine 2000 (Life Technologies) according to standard methods (Ludwig et al. J Biol Chem 276, 10990-10998, 2001). The transfection efficiencies were over 60%. 24 hours after the transfection, an infection with the influenza A virus strain fowl plague virus A/Fpv/Bratislava/79 was performed with a multiplicity of infection of 1 (MOI=1). Another 24 hours after the infection, the titers of the newly formed viruses in the cell culture supernatant were tested in standard plaque assays for MDCK cells. The virus titers of influenza A virus-infected MDCK cells infected with either the empty vector or a construct, which expressed the dominant-negative form of SEK, were compared.

Further, by means of retroviral transduction, MDCK cell lines were produced, which stably expressed either SEK KD or an SEK antisense RNA. An overexpression of the dominant-negative kinase SEK KD inhibits competitively the corresponding wild type, whereas by generation of an RNA species (antisense RNA) being complementary to the SEK/MKK4 messenger RNA, the synthesis of endogenous SEK/MKK4 is inhibited. For generating these stable MDCK cell lines, the cDNA for SEK KD was cloned in sense as well as antisense orientation into the retroviral expression vector pCFG5 IEGZ (Kuss et al. Eur J Immunol 29, 3077-3088, 1999). In addition to the messenger RNA for SEK KD or the antisense RNA, the vector DNA further codes for the messenger RNA of the green fluorescent protein (GFP), which is expressed during the protein synthesis, starting from an internal ribosome binding site. This permits the identification of stably transfected cells in the flow cytometry. Furthermore, the vector mediates a resistance against the antibiotic Zeocin. The expression constructs for SEK KD and SEK-antisense RNA as well as the empty vector were transfected by means of the calcium phosphate precipitation method into the virus-producing cell line ØNX (Grignani et al. Cancer Res 58, 14-19, 1998). The transfection efficiency was checked after 24 hours based on the GFP expression, and was in the order of 70-80%. The cells were then selected for approx. two weeks with 1 mg/ml Zeocin in the medium. For the infection of MDCK cells with the recombinant retroviruses, the retrovirus-containing medium supernatants of the virus-producing cell lines were filtrated, reacted with 5 μg/ml Polybrene (Sigma-Aldrich, Taufkirchen near Munich, Germany) and given on fresh MDCK cells. The infection took place twice on two successive days during centrifugation (1,000 g) of 3 hours. Stably transducted MDCK cells were selected 24 hours after infection for two further weeks with 400-600 μl/ml Zeocin in the medium supernatant. After the thus made generation of the stable cell lines, these as well as wild type MDCK cells were infected with influenza A virus, and the virus titers of SEK KD and SEK antisense lines were determined as described above in comparison to the titer from the supernatant of wild type MDCK cells. The following results were obtained. The comparison of the virus titers of influenza A virus-infected MDCK cells, which previously have been transfected either with the empty vector or a construct expressing the dominant-negative form of SEK, showed that in SEK KD-expressing cells, the multiplication of the viruses was inhibited after 24 hours by more than 90%. This result could be reproduced in several independent sequences. For further investigation, MDCK cell lines, which stably expressed either SEK KD or an SEK antisense RNA (see above), were produced by means of retroviral transduction. In the one case, the SEK/MKK4 activity is inhibited by competition, in the other case, expression inhibition of the kinase takes place. In these two cell lines, too, the virus titers were strongly reduced 24 hours after infection with two different influenza A virus strains (FPV and WSN-HK), compared to the wild type cells, which shows that the blocking of the virus-activated signal transmission path on the level of SEK has a decisive effect on the virus multiplication. These results show that the specific inhibition of the superordinated SEK kinase within the cellular MEKK/SEK/JNK signal transmission path inhibits the virus multiplication, and this at least as effectively as the kinase inhibitor U0126, which is known to inhibit the signal transmission via Raf/MEK/ERK by inhibition of MEK. Furthermore, the possibility to generate cell lines overproducing SEK KD and SEK antisense RNA, which with regard to their morphology and their growth behavior cannot be differentiated from vector cells or wild type MDCK cells, shows that the specific inhibition of function or expression of this kinase is not toxic for the host cell.

### EXAMPLE 4

### Combination of Two Active Substances for the Inhibition of the Virus Multiplication for the Cellular Raf/MEK/ERK Signal Transmission Path

The specific effect of the inhibition of two kinases within a cellular signal transmission path, which can be activated one immediately after the other, on the influenza virus multiplication was investigated for the example of the Raf/MEK/ERK signal transmission path. For this purpose, by means of the transient transfection, on the one hand a dominant-negative mutant of the kinase Raf (RafC4B), and on the other hand dominant-negative mutants of the kinase ERK2 (ERK2C3, ERK2B3) were brought into MDCK cells for the inhibition of the respective wild type kinase and were overexpressed. RafC4B is a deletion mutant of Raf, which lacks the kinase domain (Bruder et al Genes Dev 6:545-556, 1992). Thereby the activating signal on the Raf level is interrupted. ERK2B3 is a point mutant of the kinase ERK2, wherein a conserved lysine in the ATP binding site at amino acid position 52 of the protein is transformed by specific mutagenesis on the DNA level into an arginine amino acid residue, a mutation, which disturbs the ATP binding of the kinase, and the kinase thus exists in an inactive form (Robbins et al J Biol Chem 268:5097-5106, 1993). In the case of ERK2C3, a tyrosine amino acid residue at position 185 of the protein in the sequence motive threonine-glutamine acid-tyrosine, which is phosphorylated in the course of the activation of the kinase, is transformed into phenylalanine, whereby the kinase cannot be activated anymore (Robbins et al J Biol Chem 268:5097-5106, 1993). ERK2C3 therefore correspondingly acts in the case of an overexpression in a competitively inhibiting manner on the endogenous wild type. For the experiment, MDCK cells were transfected with the empty vector KRSPA or with the expression constructs KRSPA RafC4B, KRSPA ERK2B3 or KRSPA ERK2C3 by means of the transfection reagent Lipofectamine 2000 (Life Technologies/Invitrogen, Karlsruhe, Germany) according to standard methods (Ludwig et al. J Biol Chem 276, 10990-10998, 2001). The transfection efficiencies were higher than 60%. 24 hours after the transfection, in part of the batches the cells for the additional inhibition of MEK in the signal transmission path were treated with 30 μM U0126, as already described above (Pleschka et al. Nat. Cell Biol. 3, 301-305, 2001). Thereafter, for all batches, the infection with the influenza A virus strain fowl plague virus A/Fpv/Bratislava/79 (H₇N₇) with a multiplicity of infection of 1 (MOI=1) was made. Another 9 hours or 24 hours after the infection, the titers of the newly formed viruses in the cell culture supernatant were tested in standard plaque assays for MDCK cells. Compared were the virus titers of: 1. infected MDCK cells, which were transfected with the empty vector, 2. infected MDCK cells, which were transfected either with RafC4B, ERK2B3 or ERK2C3, 3. infected MDCK cells, which were transfected with the empty vector and additionally treated with U0126, and 4. infected MDCK cells, which were transfected either with RafC4B, ERK2B3 or ERK2C3 and additionally treated with U0126. The following results were obtained. The comparison of the virus titers of influenza A virus-infected MDCK cells, which previously have been transfected either with the empty vector or constructs, which expressed dominant-negative forms of RafC4B, ERK2B3 or ERK2C3, showed that in cells, which expressed the kinase mutants, the virus multiplication was significantly inhibited after 9 hours and 24 hours. The same applies to the virus multiplication in cells, which were transfected with the empty vector and treated with U0126, compared to cells not treated with U0126. I-f in cells, in which Raf or ERK was inhibited by overexpression of RafC4B or ERK2B3 or ERK2C3, according to the invention the kinase MEK as a second kinase is in addition inhibited within the signal transmission path, an increased inhibition of the virus multiplication is found, compared to the batches, which have not been subjected to an additional inhibition of the kinase MEK by U0126. This shows that the inhibition of two kinases within a signal transmission path inhibits the virus multiplication to a larger extent, than this is possible by inhibition of only one kinase within the signal transmission path.

### EXAMPLE 5

### Specific Inhibition of the Virus Multiplication for the Cellular Raf/MEK/ERK Signal Transmission Path

The specific effect of the inhibition of kinases within a cellular signal transmission path on the influenza virus multiplication was investigated for the example of the Raf/MEK/ERK signal transmission path. For this purpose, by means of the transient transfection, on the one hand a dominant-negative mutant of the kinase Raf (RafC4B), and on the other hand dominant-negative mutants of the kinase ERK2 (ERK2C3, ERK2B3) (see above) were brought into MDCK cells for the inhibition of the respective wild type kinase and were overexpressed. For the experiment, MDCK cells were transfected with the empty vector KRSPA or with the expression constructs KRSPA RafC4B, KRSPA ERK2B3 or KRSPA ERK2C3 by means of the transfection reagent Lipofectamine 2000 (Life Technologies/Invitrogen, Karlsruhe, Germany) according to standard methods (Ludwig et al. J Biol Chem 276, 10990-10998, 2001). The transfection efficiencies were higher than 60%. After 24 hours, in all of the batches infection with the influenza A virus strain fowl plague virus A/Fpv/Bratislava/79 (H₇N₇) with a multiplicity of infection of 1 (MOI=1) was made. Another 9-24 hours after the infection, the titers of the newly formed viruses in the cell culture supernatant were tested in standard plaque assays for MDCK cells. Compared were the virus titers of: 5. infected MDCK cells, which were transfected with the empty vector, 6. infected MDCK cells, which were transfected either with RafC4B, ERK2B3 or ERK2C3. The following results were obtained. The comparison of the virus titers of influenza A virus-infected MDCK cells, which previously have been transfected either with the empty vector or constructs, which expressed dominant-negative forms of RafC4B, ERK2B3 or ERK2C3, showed that in cells, which expressed the kinase mutants, the virus multiplication was significantly inhibited. This showed that the inhibition of different kinases within a signal transmission path can represent the starting-points for the inhibition of the virus multiplication.

### EXAMPLE 6

### Combination of Two Active Substances for the Inhibition of the Virus Multiplication for the Cellular MKK6/p38/3pK Signal Transmission Path

The specific effect of the inhibition of two kinases within a cellular signal transmission path, which can be activated one immediately after the other, on the influenza virus multiplication was further investigated for the example of the cellular MKK6/p38/3pK signal transmission path. For this purpose, by means of the transient transfection, on the one hand a dominant-negative mutant of the kinase MKK6 (MKK6(Ala)), and on the other hand a dominant-negative form of the kinase 3pK (3pK K>M), a kinase substrate of the p38 MAP kinase, was brought into MDCK cells for the inhibition of the respective wild type kinase and was overexpressed. 3pK K>M is a point mutant of the kinase 3pK, wherein a conserved lysine in the ATP binding site at amino acid position 73 of the protein was transformed by specific mutagenesis on the DNA level into an methionine (Sithanandam et al. Mol Cell Biol 16:868-876, 1996), a mutation, which disturbs the ATP binding of the kinase, and the kinase thus exists in an inactive form. In the case of MKK6(Ala), correspondingly the lysine in the ATP binding site at amino acid position 82 of the protein was transformed by specific mutagenesis on the DNA level into an alanine (Raingeaud et al Mol Cell Biol 16:1247-1255, 1996). By this inactivation, MKK6(Ala) acts in the case of an overexpression in a competitively inhibiting manner on the endogenous wild type. For the experiment, MDCK cells were transfected with the empty vector KRSPA or with the expression constructs KRSPA MKK6(Ala) or 3pK K>M by means of the transfection reagent Lipofectamine 2000 (Life Technologies) according to standard methods (Ludwig et al. J Biol Chem 276, 10990-10998, 2001). The transfection efficiencies were higher than 60%. 24 -hours after the transfection, in part of the batches the cells for the additional inhibition of the kinase in the signal transmission path were treated with 20 μM SB202190, a specific inhibitor of the p38 MAP kinase (Cohen Trends Cell Biol 7:353-361, 1997). Thereafter, for all batches, the infection with the influenza A virus strain fowl plague virus A/Fpv/Bratislava/79 with a multiplicity of infection of 1 (MOI=1) was made. Another 9 hours or 24 hours after the infection, the titers of the newly formed viruses in the cell culture supernatant were tested in standard plaque assays for MDCK cells. Compared were the virus titers of: 1. infected MDCK cells, which were transfected with the empty vector, 2. infected MDCK cells, which were transfected either with MKK6(Ala) or 3pK K>M, 3. infected MDCK cells, which were transfected with the empty vector and additionally treated with SB202190, and 4. infected MDCK cells, which were transfected either with MKK6(Ala) or 3pK K>M and additionally treated with SB202190. The following results were obtained. The comparison of the virus titers of influenza A virus-infected MDCK cells, which previously have been transfected either with the empty vector or constructs, which expressed dominant-negative forms of MKK6 or 3pK, showed that in cells, which expressed these kinase mutants, the virus multiplication was significantly inhibited after 9 hours and 24 hours. The same applies to the virus multiplication in cells, which were transfected with the empty vector and treated with SB202190, compared to cells not treated with SB202190. If in cells, in which MKK6 or 3pK was inhibited by overexpression of MKK6 (Ala) or 3pK K>M, according to the invention the kinase p38 as a second kinase is in addition inhibited within the signal transmission path, an increased inhibition of the virus multiplication is found, compared to the batches, which have not been subjected to an additional inhibition of the kinase p38 by SB202190. This shows that the inhibition of two kinases within a cellular signal transmission path inhibits the virus multiplication to a larger extent, than this is possible by inhibition of only one kinase within the signal transmission path.

### EXAMPLE 7

### Effect of the Combination of Amantadine and U0126 on the Influenza Virus Infection

It has been shown by Scholtissek and Müller (Arch. Virol. 119:111-118, 1991) that the treatment of influenza-infected cells with a combination of a methylation inhibitor (3-deazaadenosine) and a broadly effective toxic kinase inhibitor (1-(5-isoquinolinesulfonyl)-2-methyl-piperazine=H7) multiplies the effect of the two individual inhibitors. It was tested whether the combination of an active substance according to the invention with an antiviral substance, which inhibits a viral component and is no kinase inhibitor, has a synergistic antiviral effectivity. Permissive MDCK cells were infected with a multiplicity of infection (MOI) of 0.01 with influenza A viruses (A/FPV/Bratislava (H7N7) and A/WSN-HK (H3N1)) and influenza B viruses/Massachusetts/6/92 (B/Mass). The infected cells were treated as follows: 1) untreated (for FPV, WSN-HK and B/Mass), 2) U0126 in optimally antivirally effective concentrations (Pleschka et al Nature Cell Biol 3:301-305, 2001) (for FPV, WSN-HK and B/Mass), 3) amantadine in optimally antivirally effective concentrations (Hay et al EMBO J. 11:3021-3024, 1985) (for FPV), 4) combinations of amantadine and U0126 (for FPV). Amantadine is known i) to be effective in pharmacologically reasonable (micromolar) concentrations only for influenza A infections, not however for influenza B infections (Davies et al Science 144:862-863, 1964), ii) to be not effective for all influenza viruses of the sub-type A, for instance not for A/WSN/33 (Thomas et al J. Virol. 252, 54-64, 1998) or A/Puerto Rico/8/34 (Castrucci et al J Virol 69:2725-8, 1995), iii) to initially strongly inhibit sensitive influenza A viruses, and to subsequently lead to the generation of resistant virus variants (Hay et al EMBO J. 11:3021-3024, 1985). 48 hours after the infection, the titer of infectious virus particles in the cell culture supernatant was determined. The supernatants of untreated cells and of cells treated with amantadine and/or U0126 were diluted 1:1,000 (the supernatants of influenza A/WSN-HK and influenza B virus-infected cells, which have been treated with U0126, were diluted 1:100), and again used for the infection of fresh cells. These infection sequences were repeated twice. The following result was obtained: i) already after the second infection sequence, in case of a treatment with optimum concentrations of U0126, a significant reduction of the influenza virus particles could be recorded, which in one test was so significant that there were not left enough infectious influenza A/WSN-HK or influenza B virus particles to permit a third infection sequence, ii) the influenza A/FPV virus titer remained continuously low, in case of a treatment with an optimum concentration of U0126, although this virus sub-type multiplies considerably faster in a cell culture than for instance influenza A/WSN-HK, iii) the titer of the amantadine-sensitive influenza A/FPV virus dropped after addition of amantadine and then increased again, which indicates the generation of resistant virus variants, iv) the titer of the not-amantadine-resistant influenza A/WSN_HK virus increased, in spite of the amantadine treatment, what confirms that not all influenza A viruses react on amantadine, v) the treatment with amantadine and an optimally antivirally effective concentration of U0126 leads to an increased inhibition of the virus multiplication for FPV. Thus the results prove the superior antiviral effect of an active substance according to the invention, which is effective against various influenza A viruses (being in part amantadine-resistant) as well as against influenza B viruses (being amantadine-resistant), and no resistance development has been observed neither for influenza A viruses nor influenza B viruses, and wherein a synergistic effect in the combination of an active substance according to the invention with an antivirally effective active substance, which is no kinase inhibitor, on influenza A viruses is shown.

### EXAMPLE 8

### Resistance Generation

This example shows that by action of the MEK inhibitor U0126 with multiple passaging, no resistant variants of influenza A and B viruses are formed. For the multiplication of influenza A and B viruses, permissive, eukaryotic cell cultures (madine darby canine kidney (MDCK) cells) are washed with a physiologic salt solution in parallel batches having identical cell counts according to methods being generally usual for cell cultures, and are infected with an amount of the infectious influenza A virus strain fowl plague virus A/Fpv/Bratislava/79 (H₇N₇) or of the infectious influenza B virus strain Massachusetts/6/93 in a ratio of 0.01 infectious virus particle per cell for one hour at room temperature. After the infection, the inoculum is removed, and the infected cells are incubated in a suitable cell culture medium, which is reacted with the MEK inhibitor U0126 (50 μM dissolved in DMSO), for 48 h at 37° C. and 5% CO₂ concentration. As a solvent control, infected MDCK cells are incubated with cell culture medium, which is reacted with the corresponding amount of DMSO. For the influenza B virus infection, the cell culture medium contains 2 μg/ml trypsin. For the influenza A virus infection, the cell supernatant was harvested after 48 hours. For the influenza B virus infection, 48 hours after the infection the same amount of inhibitor or solvent was again added. The cell supernatant was harvested after another 24 hours. 0.1 ml of a 10−2 dilution (influenza B virus) or a 10−3 dilution (influenza A virus) of the cell supernatants was used for the infection of fresh MDCK cells (passage). The protocol was repeated four times, and after every cycle the virus titer was determined. The samples of the respective cell culture supernatants are investigated according to usual virological methods for the amount of newly formed infectious virus particles (plaque assay on MDCK cells). In an additional check, FPV-infected MDCK cells were treated with amantadine (5 μM, cell supernatants were harvested 48 hours after the infection and used in 0.1 ml of a 10−3 dilution for passaging), which significantly inhibits the multiplication of some influenza A viruses, since it inhibits the ion channel activity of the M2 protein. With regard to amantadine, reference is made to example 7. As a result, it can be stated in such an experimental approach that with the corresponding concentration of the MEK inhibitor U0126 in the cell culture medium, a significant and constant reduction (approx. 80%) of the number of newly formed infectious influenza A virus particles, compared to the solvent control without MEK inhibitor U0126, will take place, without resistant virus variants being formed, which would lead to an increase of the virus titer, as can clearly be seen in the case of the amantadine treatment. Furthermore, a significant and constant reduction (approx. 90%) of the number of newly formed infectious influenza B virus particles, compared to the solvent control without MEK inhibitor U0126, will also be found, without resistant virus variants being formed, which would lead to an increase of the virus titer.

### EXAMPLE 9

### Negative Control

This example shows that by action of the transient expression of constitutively active forms of the kinases Raf (Raf BXB, deletion mutant, which contains the kinase domain only, described in: Flory, E., Weber, C. K., Chen, P., Hoffmeyer, A., Jassoy, C. & Rapp, U. R. (1998) J. Virol. 72, 2788-2794) and MEK (delta stu MEK, formed by stu-mediated deletion of an inhibitory alpha helix, described in: Apoptosis suppression by Raf-1 and MEK1 requires MEK and phosphatidylinositol 3-kinase-dependent signals, by Gise A, Lorenz P, Wellbrock C, Hemmings B, Berberich-Siebelt F, Rasp U R, Troppmair J. Mol Cell Biol 2001 April; 21(7):2324-36) in MDCK cells, the multiplication of influenza A viruses is significantly increased. For the multiplication of influenza A viruses, permissive, eukaryotic cell cultures MDCK cells are transfected in parallel batches having identical cell counts, according to methods being generally usual for the cell culture, with plasmid DNA of the corresponding expression plasmids or of the vector control (see above). The transfection efficiencies were approx. 80%. 24 hours after the transfection, the cells are washed with a physiologic salt solution according to methods being generally usual for the cell culture, and are infected with an amount of the infectious influenza A virus strain fowl plague virus A/Fpv/Bratislava/79 (H7N7) in a ratio of 1-10 infectious virus particles per cell for 1 hour at room temperature. Then the inoculum is replaced by a suitable cell culture medium. After another 24 hours, the number of newly formed virus particles was determined by plaque assay (see above). As a result, it can be stated in such an experimental approach that the number of newly formed infectious virus particles of MDCK cells, which prior to the virus infection have been transfected with the corresponding expression plasmids, significantly increases for influenza A viruses, compared to MDCK cells, which prior to the virus infection have been transfected with the vector control only.

1. A method for the prophylaxis and/or treatment of at least one viral disease, comprising:
administering a pharmaceutical composition comprising at least one active substance wherein the active substance(s) inhibit(s) either at least two kinases or at least one SEK kinase of a cellular signal transmission path such that a virus multiplication is inhibited.

2. The method of claim 1, wherein the kinases of the cellular signal transmission path can be activated one immediately after the other.

3. The method of claim 1 wherein the kinases or the SEK kinase of the cellular signal transmission path are or is respectively selected from the following signal transmission paths: MEKK2,-3/MEK5/ERK5; Raf/MEK/ERK; MEKK/SEK/JNK; JAK1, JAK2, JAK3, TYK2 and/or hetero and homodimers of JAK1,-2,-3, TYK2; ASK/MKK3, -6/p38; ASK/MKK4,-7/JNK; MEKK4/MKK4,-7; DLK/MKK4,-7; Tpl-2/MKK4,-7; Tpl-2/MEK5/ERK5; MLK-3/MKK3,-6; MLK-3/MKK4,-7; TAK/NIK/IKK; TAK/MKK3,-6; TAK/MKK4,-7; PAK/MKK3,-6; PAK/IKK; Cot,Tpl-2/IKK; PKC/IKK; PKB/IKK; PKC/Raf; PAK/Raf; Lck/Raf; MEKKs/IKK; PI3K/PDK1/PKB; JAK/TYK/PLCgamma; and MAP kinases/MAPKAP kinases, comprising ERK/3pK, ERK/Rskp90, p38/MAPKAP kinases or p38/3pK.

4. The method of claims 1 or 3, wherein the active substance(s) is or are, respectively, selected from the following active substances: kinase-inhibiting flavone derivative or benzopyran derivative; kinase inhibiting derivative of the 4H-1-benzopyran; flavopiridol derivative; 2-(2-amino-3-methoxyphenyl)-4-oxo-4H-(1) benzopyran; 7,12-dihydro-indolo(3,2-d)(1)benzazepin-6(5H)-on; 70H-staurosporine or a phosphokinase-inhibiting derivative of the 70H-staurosporine; butyrolactone; roscovitine; purvalanol A; emodin; anilinoquin-azoline; phenylaminopyrimidine; trioylimidazole; paullone; [4-(4-fluorophenyl)-2-(4-methylsulfinylphenyl)-5-(4-pyridyl) 1H-imidazole; [1,4-diamino-2,3-dicyano-1,4-bis(2aminophenylthio) butadiene; kinase-inhibiting derivative of the butadiene; [2-2′-amino-3′-methoxyphenyl)-oxa-naphthalene-4-on); [2-(2-chloro-4-iodo-phenylamino)-N-cyclo-propylmethoxy-3,4-difluoro benzamide; CEP-1347 (KT7515); bis-ethylthiomethyl; tetrapyrrolic macrocycles; pyrimidone derivative; 3-aminomethylen-indoline derivative; pyrazolo (3,4-b) pyridine derivative; pyrazole derivative; 1,4-substituted piperidine derivative; lipoid ammonium salt; dominant-negative mutant of a kinase of a cellular signal transmission path; antisense oligonucleotide, which specifically adds to the DNA sequence or mRNA sequence coding for a kinase of a cellular signal transmission path and inhibits the transcription or translation thereof; ds-oligonucleotides, which are suitable for a specific degradation of the mRNAs of kinases of a cellular signal transmission path by the RNAi technology; antibodies or antibody fragments specific for a kinase or a fusion protein, containing at least one antibody fragment, comprising an Fv fragment which inhibits the kinase activity of a kinase module; and peptide, which inhibits the interaction of at least two kinases, which can be activated one immediately after the other, of a cellular signal transmission path.

5. The method of claims 1 or 4, wherein the viral disease is caused by RNA or DNA viruses.

6. A combination preparation for the prophylaxis and/or treatment of at least one viral disease, containing at least two active substances, which act either on at least two kinases of a cellular signal transmission path such that a virus multiplication is substantially inhibited, or substantially inhibit an SEK kinase, wherein the combination preparation can be used in the form of a mixture or as separate components for the application at the same time or at different times at the same position or at different positions.

7. A combination preparation for the prophylaxis and/or treatment of at least one viral disease, containing at least one active substance according to claims 1 or 6 and at least one antivirally effective substance, which is not a kinase inhibitor.

8. A combination preparation according to claim 7, wherein the antivirally effective substance, which is not a kinase inhibitor, is 1-adamantanamine, rimantadine, a neuramimidase inhibitor or a nucleoside analog comprising ribavirin.

9. An active substance or a combination preparation according to claim 1 for the prophylaxis and/or treatment of an infection with negative strand RNA viruses, comprising influenza viruses or Borna viruses.

10. A test system for finding active substances, which act on at least two kinases or on an SEK kinase of a cellular signal transmission path such that a virus multiplication is substantially inhibited, a) comprising at least one cell wherein the cell is injectable with at least one virus, which contains either at least two kinases of a cellular signal transmission path or at least one SEK kinase and at least one virus infecting the cells, or b) the cell is infected with at least one virus, which contains either at least two kinases of a cellular signal transmission path or at least one SEK kinase.

11. A test system according to claim 10, wherein the virus is an RNA or DNA virus comprising an influenza virus.

12. A test system according to claim 10 wherein the cell contains at least one overexpressed kinase.

13. A test system according to claim 10 wherein the cell further comprises a) at least one gene coding for at least one dominant-negative mutant of at least one superordinated kinase or b) at least one dominant-negative mutant of at least one subordinated kinase.

14. A test system according to claims 10 wherein the expression of at least one kinase is inhibited.

15. A method for finding at least one active substance for the prophylaxis and/or treatment of viral diseases, which substantially inhibit(s) the multiplication of viruses in viral diseases, comprising the following steps: a) bringing at least one test system according to claim 10 into contact with at least one potential active substance, and b) determining the effect on virus multiplication.

16. A method for producing a drug for the prophylaxis and/or treatment of at least one viral disease, which substantially inhibit(s) the multiplication of viruses in viral diseases, comprising the following steps: a) performing a test system according to claim 10, and b) reacting the found active substance(s) with at least one auxiliary and/or additional substance.

17. A method for the prophylaxis and/or treatment of viral diseases comprising administering a pharmaceutical composition comprising one or several of the following active substances:
a) an inhibitor of a kinase, the inhibition of which inhibits virus multiplication, in combination with an antiviral active substance, which is no kinase inhibitor, b) an inhibitor of a first kinase, the sole inhibition of which inhibits virus multiplication, and the inhibitor in addition inhibits a second kinase different from the first kinase, and the sole inhibition of the second kinase inhibits virus multiplication, c) a first inhibitor of a first kinase, the sole inhibition of which inhibits virus multiplication, in combination with a second inhibitor of a second kinase different from the first kinase, and the sole inhibition of the second kinase inhibits virus multiplication, the second inhibitor being different from the first inhibitor, or d) an inhibitor of an SEK kinase.

18. A method for screening for prospective active substances of claim 17, wherein a prospective active substance is contacted with a test system according to claims 10, wherein the inhibition of virus multiplication is quantified, wherein the obtained value of the inhibition is compared to a reference value of the inhibition, which is obtained under identical conditions without a prospective active substance or a reference active substance or a reference active substance combination, and wherein a prospective active substance or a prospective active substance combination is selected, if the value of the inhibition is higher than the reference value of the inhibition.

19. A method for the prophylaxis and/or treatment of a viral disease, wherein a pharmaceutical composition according to claim 17 is administered to a patient in a defined and physiologically effective dose.

20. The method of claim 5, wherein the viral disease is caused by influenza viruses.

21. The preparation of claim 6, wherein the active substance(s) is selected from those according to claim 3.

22. The method of claim 18, wherein a prospective active substance and a previously identified different active substance or different further prospective active substance is contacted with the test system according to claim 10.
